(19) United States
(12) Patent Application Publication
(10) Pub. No.: US 2006/0189967 A1
(43) Pub. Date: Aug. 24, 2006

(54) Device, a catheter, and a method for the curative treatment of varicose veins

(76) Inventors: Leonardo Masotti, Firenze (IT); Antonio Collarino, Pistola (IT); Damiano Fortuna, Firenze (IT); Cesare Paolini, Firenze (IT)

Correspondence Address: MCGLEW & TUTTLE, PC, P.O. BOX 9227, SCARBOROUGH STATION, SCARBOROUGH, NY, 10510-9227, US

(21) Appl. No.: 11/136,262

(22) Filed: May 24, 2005

(30) Foreign Application Priority Data
Feb. 21, 2005 (IT) FI2005A000028

(51) Int. Cl. A61B 18/18

(52) U.S. Cl. 606/15; 606/9; 606/3; 606/17

(57) ABSTRACT

Described herein are a device and a method for the treatment of varicose veins via laser radiation, and in particular using a holmium laser. The radiation of a laser source (5) is injected in a fiber (3) that can be inserted in the vessel to be treated. The laser source emits a radiation such as to cause a hyalinizing sclerosis with structural modifications both to the fibers of the collagen (shrinkage) and to the extracellular matrix of the median coat of the vein by the photothermal effect, substantially without thermal stress of the morphological component of the tunica media and of the tunica intima.

### TECHNICAL FIELD

The present invention relates to innovations in the field of treatment of varicose veins. More in particular, the present invention relates to a particular and innovative catheter, which can be used for this type of treatment, as well as to a device or apparatus for treatment and to a method of treatment.

### PRELIMINARY REMARKS AND STATE OF THE ART

Varicose veins represent one of the most common chronic pathological conditions that evolve in such a way as to require surgical intervention. It is a pathological condition that is typical of the more advanced nations and one which has a considerable socio-economic impact. It presents, in fact, a marked prevalence, amounting to approximately 10% of the population. In the USA, for example, there are approximately 25,000,000 people affected by varicose veins, and of these some 2,500,000 suffer from chronic venous insufficiency (CVI), whilst 500,000 have ulcerative lesions.

There is a greater prevalence of varicose veins in females (50-55%) than in males (10-50%), with annual rates of incidence of 2.9% and 1.6%, respectively. As regards the age range, a higher incidence is found in adults than in young people, with peaks of up to 78% in patients over 60 years of age.

It is an orthostatic pathological condition. Amongst the predisposing factors there figures above all that of familial risk, which would seem to be more of a phenotypic nature (e.g., obesity) than a genotypic one. Amongst risk factors it is possible to number: pregnancy; occupations that require prolonged standing; obesity; and physical inactivity.

The peripheral venous network, both superficial and deep, is a system with low efficiency and limited capacity for compensation. The venous wall has a low degree of elasticity and reactivity to the parietal centrifugal forces. In the physiological context, this characteristic enables the venous vessels, by dilating, to function as decompression chambers, thus offering the right contribution to the systemic hemodynamic balance. On the other hand, the reduced vicarious capacity (i.e., that of compensation) with which the venous system is equipped limit reactivity to chronic tension of the wall, which, thus stimulated, tends to undergo a progressive wear. This same mechanism also involves the valvular system. With the onset of valvular incompetence there arise mechanisms of reflux, with reversal, to a varying degree, of the venous flow. From this stage on, the phenomenon tends to become irreversible, and the only therapeutic perspective existing today consists in the functional exclusion of the lesioned stretch, and hence an intervention of a “destructive” type. This may be an intervention of a destructive nature proper, which contemplates the surgical removal of all or part of the diseased vein, or else a destructive intervention in a functional sense, which contemplates its permanence in situ after obliteration. For the first type we shall use the term “anatomical destructive intervention”, whilst for the second “functional destructive intervention”.

The extreme complexity of the anatomical structure of the venous network of the lower limb, the individual pleomorphism, as well as the ample physiopathological variability of the varicose lesion, render it difficult to arrive at a schematization of the condition. It is consequently even more surprising that the therapeutic approach has been for at least one century to the present day, namely, at least until the development of the techniques known as CHIVA (cure Conservatrice et Hémodynamique de l'Insuffisance Veineuse en Ambulatoire), which will be discussed in greater detail hereinafter, reducible to a single scheme: anatomical and/or functional exclusion of the affected area.

Anatomical and/or functional exclusion of the affected area in effect takes the form of a destructive form of treatment.

Unfortunately, this approach does not envisage the correction of the hemodynamic disorders, which constitute the source of varicose veins, but by reducing the vasal network, it paradoxically contributes to reducing the possibility of discharge of pressure and venous efflux, so aggravating, over time, a situation that is already insufficient. It is for this reason that this approach involves high rates of recidivation. Such rates vary over time in the range comprised between a minimum of 20% at 6 months up to a maximum of 60-80% at 2 years.

Up to approximately ten years ago, surgery and sclerotherapy represented the main, and indeed almost exclusive, therapeutic procedures. Both of the methods were proposed at the start of the last century and over the years have undergone procedural, but not substantial, improvements. The very first procedures of surgical treatment envisaged the removal of the stretches of dilated vessel. In order to prevent the drawbacks deriving from the removal of the portions of vein, surgical instruments have been produced which can be inserted via a catheter into the vein and are designed to remove the endothelium, i.e., the innermost layer of the intima of the vein, to bring about obliteration of the vein itself. An example of a surgical instrument of this type is described in the U.S. Pat. No. 5,011,489.

In U.S. Pat. No. 5,658,282 there is, instead, described a surgical instrument for making a bypass in the vein and destroying the damaged valves. A further device for executing the bypass of the damaged vein is described in U.S. Pat. No. 6,267,758.

The U.S. Pat. No. 5,695,495 describes a catheter for sclerotherapy, comprising an electrode that is inserted into the area to be treated via a pervious needle. The area treated is destroyed via the heat generated by the passage of electrical energy. A further device of this type is described in U.S. Pat. No. 6,293,944.

In the last ten years alternative, less invasive, procedures of the same method have been introduced, namely destructive surgery, via the use of diode lasers or radio-frequency apparatuses.

U.S. Pat. No. 6,033,398 describes a catheter provided with radio-frequency electrodes, used for local heating of the vessel wall and for causing a local restriction of the vein in a position corresponding to a venous valve, for the purpose of restoring at least in part the functions thereof. The heating, which can be obtained also using other energy sources, such as a laser, is limited to small areas and has only the function of restricting the vessel in an area corresponding to the valve, the functionality of which is to be recovered. Heating of a complete stretch of vessel is not envisaged.

Catheters of a similar sort are described in U.S. Pat. No. 6,036,687, U.S. Pat. No. 6,263,248, U.S. Pat. No. 6,613,045, U.S. Pat. No. 6,152,899, and U.S. Pat. No. 6,638,273. In some of these patents there are described methods of treatment to obtain functional renewal of the vein based upon an effect of coarctation, i.e., of shrinkage of the venous wall. This phenomenon, on the other hand, is described therein in altogether generic terms, and no specific reference is made to one or other of the coats (intima, media and adventitia) that form the vasal wall, nor to the possibility that the treatment applied expresses different effects on these different coats of the vessel wall. The vasal intima is a very thin membrane, formed by one or two layers of very flattened endothelial cells resting on a thin basal lamina of elastic connective tissue. Any irreversible alteration to the intima inevitably involves the formation of a microthrombus and the activation of the smooth muscle cells that migrate from the media towards the intima, following upon damage. These cells tend to proliferate and contribute, together with the initial evolution of the thrombus, to the formation of a thrombus first and of a possible stenotic plaque subsequently. If the lesions to the intima are vast, or else numerous, the microthrombi tend to converge and a stenotic evolution of the lesion is observed; in other words, there is the obliteration of the vessel. In fact, all the destructive techniques that aim at obliteration set themselves as objective the destruction of the vasal intima. In U.S. Pat. No. 6,033,398 and other subsequent ones referred to above, there are generically described catheters capable of vehicling energy sources (amongst which also laser is incidentally mentioned) to induce shrinkage of the venous wall.

However, this modality of application has not in practice yielded useful results, in so far as if the energy applied distributes uniformly, as described in these patents, on the wall of the vein, it inevitably affects and stresses also the intima of the vessel.

Although in the aforesaid patents a generic reference is also made to laser sources as possible sources of energy for the treatment of veins, there is in practice described and proposed only a radio-frequency (RF) device. It has been experimentally found that the effects of the passage of current through a biological tissue are altogether different from the effects induced by the laser on the biological tissues themselves. From a comparison between the tissue ablation induced by laser and that induced by an RF lancet, there have been observed very different effects on the tissues that are left behind in the organism.

In the case of shrinkage, there is induced a permanent modification, in the sense that the alteration induced is not resolved spontaneously but remains present for many days until the tissue thus altered is re-modeled by endogenous physiological mechanisms. Hence the context is that of “permanent”, and hence surgical, modifications.

Said effects could also be classified as primary effects, viz., those occurring immediately, and secondary effects, viz., those deferred in time.

As far as the immediate effects are concerned, both lasers and RF devices, which are both employed with surgical parameters, induce three different types of effects, distinguished into as many areas: vallum of ablation, area of permanent coagulation, and area of temporary thermal stress.

In the comparison between the laser and radio-frequency techniques, the amplitude of the three areas immediately after application depends upon many variables, even though on average with the radio-frequency technique the impact on the tissues is more profound as compared to the laser technique (above all, as compared to lasers that have high coefficients of absorption for water: CO₂, erbium and holmium lasers).

Very different, instead, is the case of effects deferred in time. In fact, with radio-frequency devices, to the aforesaid three areas there is to be added another, which could be defined as “area of passage of the induced current”. This is generally a rather extensive area, which regards the passage of current in the tissue comprised between the opposite poles. The tissue involved by the RF radiation undergoes the temporary phenomenon of reversal of the membrane potential and blockage of the sodium-potassium pump. There hence follows a phase of cellular suffering that frequently results in an intracellular edema, also referred to as “hydropic degeneration”. Usually, this is a reversible phenomenon unless the cells themselves are not simultaneously involved by a sudden thermal rise. In any case, the hydropic degeneration of an extensive portion of tissue delays by at least two weeks the natural hyperplastic-regenerative phenomena.

In the US patents referred to above, for example U.S. Pat. No. 6,036,687; U.S. Pat. No. 6,033,398 and U.S. Pat. No. 6,152,899, to obtain shrinkage of the wall, there is proposed a particular catheter. This is an exclusively intravascular catheter, constituted by a complex instrument that inevitably cannot fail to have a large diameter (typically with a minimum diameter of 2.3 mm expandable up to 15 mm), which in effect excludes its use for vessels of small caliber (i.e., ones smaller than 2.3 mm).

Said catheter has the capacity of self-expansion in such a way as to adapt to the caliber of the vessel, enabling contact of the electrodes to the intima of the vessel, to guarantee the directionality of the localization of the energy, confining the shrinkage just to the tissue comprised between the two electrodes of opposite polarity. The need to propose such a complex catheter is thus to be sought in the poor selectivity towards the target of the RF radiation.

U.S. Pat. No. 6,398,777 describes a laser device for intravascular treatment of varicose veins, in which a laser source is used to cause obliteration of the vessel. The technique is based upon the irreversible damage of the vessel wall throughout its thickness.

U.S. Pat. No. 6,402,745 describes an electrode for intravascular treatment of varicose veins, by means of which the vessel wall is heated by supply of electrical energy until it is destroyed.

There have also been used obliterative systems based upon the use of ultrasound. U.S. Pat. No. 6,436,061 describes, for example, an ultrasound generator which, applied on the outside of the limb affected by varices, in a position corresponding to the vein to be treated, supplies energy in the form of ultrasound waves that concentrate in the area to be obliterated.

In other techniques of sclerotherapy the obliteration of the vessel is obtained via insertion of sclerosant agents by means of a suitable catheter. An example of a device and of a method of this type are described in U.S. Pat. No. 6,726,674.

In summary, all these methods aim at achieving obliteration (sclerotherapy) or removal (saphenectomy) of the entire diseased vessel or of a portion thereof.

An improvement from the functional standpoint in the therapeutic approach consists in the obliteration of the saphenofemoral junction (or cross). It is in fact known that, in the case where there is valvular insufficiency with retrograde venous reflux, recidivation is certain. There has then been noted an increase in the clinical effectiveness via association of obliteration of the cross with saphenectomy. It is, on the other hand, known that sclerotherapy is not effective in vessels of large caliber and in patients with incontinence of the cross.

In conclusion, using destructive methods, both anatomical and functional ones, the aesthetic problems are eliminated, the clinical symptoms are alleviated temporarily, but the problem is not solved since such methods paradoxically contribute to reducing functionality of the organ. This inevitably cannot fail to be reflected in a high rate of incidence of relapse.

Recently, there have been proposed conservative but not curative methods. These contemplate external sapheno-femoral valvuloplasty and the hemodynamic corrections referred to as CHIVA 1 (cure Conservatrice et Hémodynamique de l'Insuffisance Veineuse en Ambulatoire) and CHIVA 2; see Tang J., Godlewsky G. et al., Morphologic changes in collagen fibers after 830 N laser welding. Lasers Surg. Med. 21(5): 438-43, 1997; and Lethias C., Labourdette L. et al., Composition and organization of the extracellular vein walls: collagen networks. Int. Angiol. 15(2): 104-13, 1996.

Whilst the success of valvuloplasty is correlated both to the integrity of the valve leaflets and to the degree of dilation of the vessel, the CHIVA method, albeit not easy to carry out, would seem to offer, as compared to the destructive approach, more guarantees in so far as it proposes correction of the hemodynamic dysfunctions, maintaining the greatest possible number of vessels pervious. The method, on the other hand, is mini-invasive but not curative.

### OBJECTS AND SUMMARY OF THE INVENTION

The object of the present invention is to provide a device and a method for the treatment of varicose veins, which will overcome totally or in part the drawbacks of the known techniques.

More in particular, the object of a particular embodiment of the invention is to provide a method and a device that will enable a conservative, mini-invasive and curative treatment, through the recovery of the tone of the venous wall.

According to a first aspect, the invention relates to an apparatus or a device for the treatment of varicose veins, comprising a laser source and at least one optical-fiber means for conveying the laser radiation either within the vein (intravascular) or outside of the vein (extravascular), in which the laser source has characteristics of emission such as to cause a hyalinizing sclerosis in the extracellular matrix of the median coat of the vein by the photothermal effect, substantially without thermal stress of the morphological component of the tunica media and of the tunica intima. In contrast with known devices, including ones of more recent conception, therefore, the device of the present invention uses a laser source, the effect of which is such as to preserve the integrity of the endothelium, and more in general of the intima (comprising the endothelium and sub-endothelium) of the treated vessel, in addition to that of the morphological component of the median coat. This enables the functional recovery of the treated vessel, instead of its demolition, whether functional or anatomical.

In practice, according to a possible embodiment of the invention, the laser source is a pulsed source and has a wavelength comprised between 800 and 2900 nm, and preferably around 2100 nm. An advantageous and preferred embodiment envisages the use, as laser source, of a holmium laser. This emits at a wavelength that has optimal characteristics of absorption. In fact, in order for the treatment to act on the structure of the median coat, the laser energy must be absorbed only on this coat of the vessel wall. In fact, with the laser radiation at 2100 nm, which is characterized by a high coefficient of absorption for the water and a low coefficient of absorption for the hemoglobin, there is the right diffusion of light through the wall of the vein. Our objective is in effect the “concentration” of the energy in the median coat of the vein, whilst the radiation should be prevented from damaging the intima or the light from diffusing beyond the venous wall itself and interacting with the structures contiguous to the vessel itself, for example artery and nerve.

The first case (i.e., damage to the intima due to radiation) occurs when wavelengths are used that have a high coefficient of absorption for water, such as the erbium laser (2900 nm), and for porphyrins (hemoglobin and myoglobin), such as lasers in the yellow and in the green. The second case (i.e., interaction with the contiguous structures) occurs, instead, when wavelengths characterized by a low coefficient of absorption for water (800-1064 nm) and high tissue penetration are used. In fact, currently the main sclerosant lasers have wavelengths comprised between the green and the red.

In the case in point, then, the laser radiation has a wavelength of around 2100 nm, which guarantees the right balance between absorption of water and absorption of porphyrins. Said radiation is vehicled via a probe located within the vessel; there also exists the possibility of carrying out extravascular treatment.

In fact, according to one possible embodiment, the device can be provided with a simple optical fiber that can be either inserted in the vein for intravascular treatment or else made to slide externally and parallel to the vein in extravascular treatment. In a preferred embodiment, on the other hand, the device comprises a catheter provided with a plurality of optical fibers terminating in one end of the catheter and arranged and made so as to orient the respective beams in a direction inclined outwards with respect to the axis of the catheter. The fibers can present a terminal portion inclined with respect to the axis of the catheter for orienting the emitted beam towards the internal wall of the treated vessel. As an alternative thereto or in combination therewith, the fibers can have a distal end machined in such a way as to orient the emitted radiation in said direction.

The terminal ends of the optical fibers are preferentially arranged according to a circular alignment to obtain a uniform distribution of the energy, and hence a uniform fluence on the internal wall of the vessel. For example, the terminal portions of the optical fibers are housed between an outer cylindrical sleeve and an inner tubular element of the catheter, which are coaxial to one another.

In order to ensure a correct treatment, according to an advantageous improved embodiment of the invention, the device comprises a plurality of thermal sensors associated to the end of the catheter. The value of temperature detected by the sensors can be used for controlling the emission of the laser source.

These sensors can advantageously be arranged on elongated elastic elements, which have a movement of extraction and retraction with respect to a terminal housing associated to the end of the catheter. Said elements can be shaped so as to bend outwards radially when they are extracted from the terminal end of the catheter. In this way the sensors are brought into contact with the internal surface of the intima, i.e., the innermost coat of the vessel wall, and can detect the temperature in a plurality of points of the wall of the vein. Typically, three sensors are used, for example three thermocouples. This arrangement enables the vein to be kept divaricated by means of the elasticity of the elements that carry the sensors and hence uniformity of irradiation to be guaranteed. Furthermore, the use of a plurality of thermal sensors enables elimination of possible wrong temperature data, due for example to a non-correct contact with the wall of the vessel or else to a malfunctioning of one of the sensors.

According to a possible embodiment of the device, the laser source is controlled in such a way as to maintain the temperature of the internal surface of the vessel below 85° C., and preferably below 65° C., and even more preferably between 45° C. and 60° C.

According to an advantageous embodiment of the invention, the laser source is pulsed at a frequency comprised between 1 and 50 Hz, and preferably between 2 and 25 Hz, and even more preferably between 5 and 20 Hz. In particular, the laser source can be pulsed at a frequency comprised between 5 and 15 Hz, and preferably between 6 and 10 Hz, and even more preferably between 6 and 8 Hz.

Advantageously, the laser source can emit at a power comprised between 0.5 and 10 W, and preferably between 1 and 8 W, and even more preferably between 1 and 5 W. The energy of each pulse emitted by the source can advantageously be comprised between 50 and 2000 mJ, and preferably between 120 and 900 mJ, and even more preferably between 150 and 700 mJ.

According to a different aspect, the invention relates to an angiological catheter for the treatment of varicose veins, comprising a plurality of optical fibers terminating in one end of the catheter and arranged and made so as to orient the respective beams in a direction inclined outwards with respect to the axis of the catheter.

Further advantageous characteristics and embodiments of the catheter according to the invention are specified in the attached claims and will be described with reference to an example of embodiment.

According to a further aspect, the invention relates to a curative method for the treatment of varicose veins, which can take the form of two different procedures:
intravascular treatment; and
extravascular treatment.

The principle of intravascular treatment and that of extravascular treatment are practically identical: the only thing that changes is the mode of introduction of the optical fiber. Extravascular treatment is performed with the individual optical fiber, whereas intravascular treatment, according to the caliber of the vessel to be treated, may be performed both with the optical fiber and using the angiological catheter in the case where it is necessary to treat vessels of large caliber (3-8 mm in diameter). The extravascular technique, instead, finds application in the treatment of vessels of small caliber and superficial vessels. In fact, thanks to the transillumination of a guide beam (He-Ne laser) these are readily visible with the naked eye by the surgeon, who can thus easily follow their subcutaneous path and consequently guide the fiber that conveys the treatment radiation. Also the parameters of treatment are similar in the two procedures, the extravascular one and the intravascular one.

The procedure of intravascular treatment, in particular, may present the following steps:
percutaneous introduction of an optical fiber into the vein to be treated;
irradiation, through said optical fiber, of the wall of said vein with laser radiation that causes a hyalinizing sclerosis, by direct photothermal effect, with structural modifications both to the collagen fibers (shrinkage) and to the extracellular matrix substantially limited to the median coat of the vein; and
sliding from above downwards (in a proximal-to-distal direction) of said optical fiber during emission of the laser radiation along the stretch of the diseased vein.

Advantageously, the wall of the vessel to be treated is impinged upon by laser radiation having a wavelength comprised between 800 and 2900 nm, and preferably around 2100 nm. Preferably, the laser radiation is pulsed with frequencies of pulsation comprised, for example, between 1 and 50 Hz, and preferably between 2 and 25 Hz, and even more preferably between 5 and 20 Hz. According to a possible embodiment of the method according to the invention, the laser radiation is pulsed at a frequency comprised between 5 and 15 Hz, and preferably between 6 and 10 Hz, and even more preferably between 6 and 8 Hz. The energy of each pulse can be comprised between 50 and 2000 mJ, and preferably between 120 and 900 mJ, and even more preferably between 150 and 700 mJ. The power of the radiation can be advantageously comprised between 0.5 and 10 W, and preferably between 1 and 8 W, and even more preferably between 1 and 5 W.

According to an advantageous embodiment, the laser radiation is dosed so as not to cause damage to the morphological component of the median coat of the treated vein and also of the intima, i.e., of the innermost coat of the vessel wall. Advantageously, the irradiation is controlled so that the temperature of the internal surface of the treated vein is kept below 85° C., and preferably below 65° C., and even more preferably is comprised between 45° C. and 60° C.

Advantageously, according to an embodiment of the method according to the invention, the laser radiation is applied for obtaining a photothermal effect that causes a coarctation (shrinkage) of the vein via breaking of the hydrogen bonds between the collagen fibers of the median coat of the vein itself. Preferably, the vein is treated with a laser radiation that creates a fibroblastic-myocellular stimulation of the median coat of the vein by the photothermal effect.

Basically, by applying the method according to the present invention, the coarctation or shrinkage of the venous wall affects exclusively the extracellular matrix and the collagen of the median coat of the vessel, without this involving the intima of the vessel itself, unlike other known methods, prevalently based upon the use of radio-frequency energy, which, albeit envisaging a curative rather than destructive aim, do not achieve the desired result.

The device used to carry out the method according to the present invention does not require contact with the vessel wall and consequently renders possible treatment only of the tunica media of the vessel, respecting the integrity of the tunica intima. This can occur thanks to the fact that the method of the present invention uses a light radiation at a precise wavelength (for example and in particular at 2100 nm), which since it has a particular coefficient of absorption in regard to the chromophores present in the area (porphyrins—myoglobin and hemoglobin—water, proteins) concentrates the energy only on the tunica media. In actual fact, the laser radiation involves both the intima and the media in very short times. Since, however, the intima does not have porphyrins (myoglobin) capable of absorbing an amount of said radiation and since it is very thin, it is, in the first place, far from receptive and above all is immediately cooled by the circulating blood. In fact, the heat absorbed is rapidly yielded to the venous blood by convection.

Instead, the median coat, constituted prevalently by myocells, has a high content in myoglobin which renders it more receptive to this specific electromagnetic radiation. The energy thus absorbed is converted into heat, which can dissipate only by conduction in so far as, at this level, there are no systems for heat exchange by convection. Said absorbed heat is, then, able to generate the phenomenon of shrinkage, modifying the structure of the collagen fibers of the media and hence its capacity for binding water.

The above situation obtains specifically in the case of intravascular treatment, i.e., conveying the laser radiation within the vein that is to be treated. On the other hand, there is a similar situation in the case of extravascular treatment. In this case, there are modifications both to the adventitia and to the media. Also here there is no involvement of the intima in so far as in this case the laser radiation does not arrive in sufficient amounts to induce thermal stress on the innermost membrane.

The laser radiation used in the method according to the present invention, for example and typically at a wavelength of 2100 nm, is far more selective towards the median coat of the vessel than is the radio-frequency radiation of the known methods extensively discussed above, and does not require particular artifices for limiting its diffusion, as is instead required for catheters based upon the use of radio frequency. In fact, with the method proposed herein, the laser radiation can be readily supplied with simple optical fibers of a diameter greater than or equal to 125 μm.

The extremely low invasiveness of the procedure underlying the method of the present invention is evident. In fact, such thin optical fibers can be easily inserted via percutaneous route, i.e., with a needle inserted in the vessel. The fiber slides easily in the vasal network without ever entering into contact with the endothelium, which thus does not undergo any kind of insult, whether mechanical, thermal, or of any other nature. There is thus also obtained the treatment of vessels of small caliber and, possibly, of vessels that are particularly delicate in so far as affected by more or less serious forms of vasculitis. Unlike the methods based upon radio-frequency radiation, moreover, the use of the laser for the curative treatment forming the subject of the present invention does not require vehicling of a fluid for cooling the vessel wall for the purpose of preventing excessive coagulation thanks to the fact that the cooling action is sufficiently supplied merely by the blood flow as device of heat exchange by convection.

Further advantageous characteristics and embodiments of the invention are indicated in the attached claims.

### BRIEF DESCRIPTION OF THE DRAWINGS

For a better understanding of the invention there now follows a description with reference to annexed drawing, which illustrates a practical embodiment of the device and of the catheter according to the invention. More in particular, in the drawing:

FIG. 1 is a block diagram of the device according to the invention;

FIG. 2 is a schematic illustration of the catheter;

FIG. 3 is an enlarged longitudinal sectional view of the distal portion of the catheter; and

FIG. 4 a front view according to the line IV-IV of FIG. 3.

### DETAILED DESCRIPTION OF THE PREFERRED EMBODIMENT OF THE INVENTION

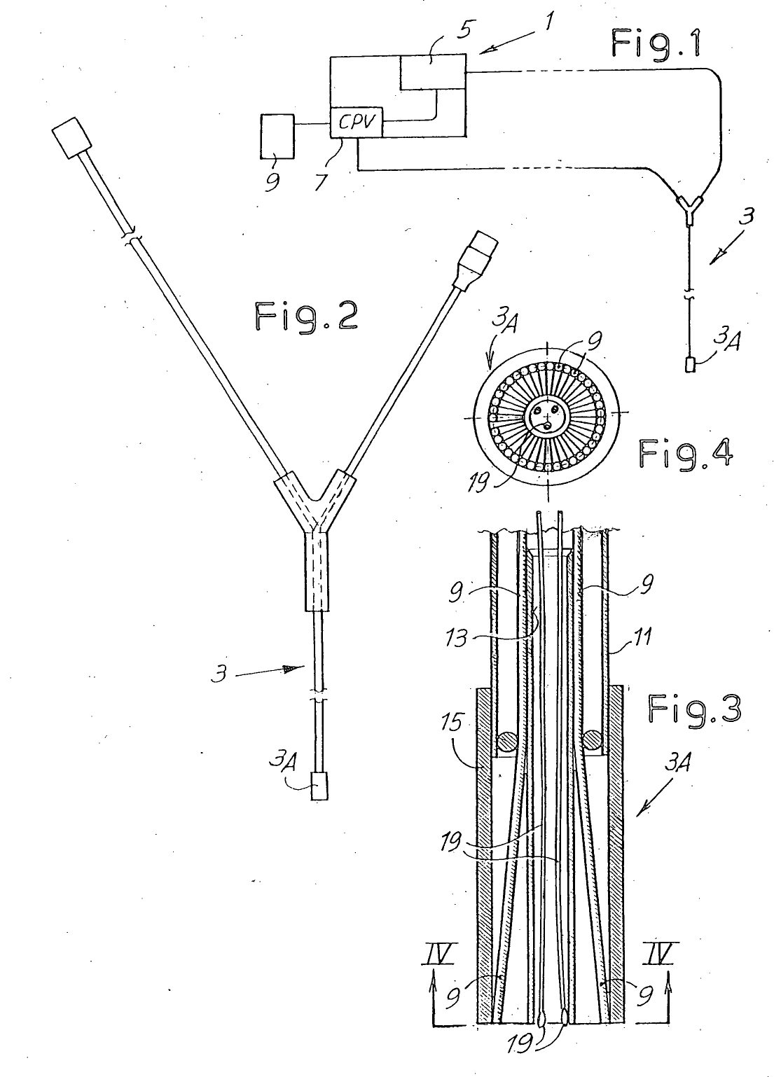

FIG. 1 is a schematic illustration of a block diagram of the apparatus according to the invention, designated as a whole by 1 and provided with at least one catheter 3, the structure of which is illustrated in greater detail in FIGS. 2 to 4. Designated as a whole by 5 is a laser source, constituted by a holmium laser, with emission at 2100 nm and pulsed at a pulse frequency of, for example, 7 Hz. The reference number 7 designates a control unit interfaced, for example, to a keyboard 9, through which the operator can set the parameters of emission of the laser.

The laser radiation emitted by the source 5 is conveyed towards the distal end 3A of the catheter 3 via a bundle of optical fibers 9 (see FIG. 3), the terminal ends of which are inclined with respect to the axis of the catheter to illuminate the vasal part. The catheter 3 comprises an outer sheath 11 and an internal tube 13. The fibers 9 pass in the space with annular cross section between the two components 11 and 13. The distal end 3A of the catheter moreover has a sleeve 15, which constitutes a prolongation of the outer sheath 9. The space between the internal tube 13 and the outer sleeve 15 is filled with epoxy resin, which seals the terminal portions of the optical fibers 9.

Within the internal tube 13, there slide elastic elements 17 made of spring steel or other resilient material, which carry at their distal ends thermal sensors 19, for example thermocouples. The elastic elements 17 are maneuverable from outside, i.e., from the proximal end of the catheter 3, in order to be extractable partially from the distal end 3A of the catheter 3. The conformation of said elements 17 is such that, when extracted from the end of the catheter, they bend outwards bringing the sensors 19 applied at their ends into contact with the vessel wall, i.e., with the intima. The number of the elements 17 can vary but is preferably at least three. They constitute in this way means for keeping the wall of the vessel divaricated and enable the optical fibers 9 to irradiate the wall itself in a uniform way. Furthermore, with at least three temperature sensors it is possible to acquire more accurate information and possibly make an average of the temperature, or else exclude a possible sensor that were to furnish a clearly erroneous value, on account of an incorrect contact with the wall of the vessel or else on account of a failure.

The catheter has (FIG. 2) a wye, which connects the optical fibers 9 coming from the source 5, and the elastic elements 17. The sensors constrained thereto are connected to the central control unit 7, which is programmed in such a way as to control emission of the source 5 so as to maintain the temperature of the vein during the treatment stage within a pre-set range of temperatures, typically between 45 and 60° C.

The treatment is prevalently, but not exclusively, intravascular with a proximal-to-distal direction. The catheter with the optical fibers 9 is inserted percutaneously, namely through a needle inserted distally in the saphena. Once the position in which the treatment is to be carried out is reached, the laser source 5 is activated, and the catheter is gradually retracted at an appropriate, and preferably constant, rate from the vein. Typically, the rate of treatment ranges between 0.5 and 3 cm/s. The variations in rate are inversely proportional to the caliber of the vessel. The fibers in this way irradiate the part of the stretch of vessel to be treated, whilst the thermal sensors detect the value of the temperature of the wall of the vessel immediately after irradiation. According to the temperature detected, it is possible to modify, either automatically or manually, the condition of emission of the laser, for example by reducing the duty cycle and/or the power and/or the frequency of the pulses. Alternatively, the control unit 7 can supply the operator, via an appropriate interface (such as, for example, a display), with information on the effective temperature of the vessel wall so that the operator himself can intervene manually to maintain the temperature within the desired values, either by intervening on the conditions of emission of the laser or, alternatively or in combination, by modifying the rate of translation of the end 3A of the catheter within the vein.

Typical conditions for the treatment of the saphena are the following:

| Degree of | Frequency | Mean power | Energy of |
|---|---|---|---|
| treatment | of pulses | of source | pulse |
| low | 7 Hz | 1.05 W | 150 mJ |
| medium | 7 Hz | 2.10 W | 300 mJ |
| high | 7 Hz | 4.9 W | 700 mJ |

It is to be understood that, rather than a catheter of the type described above, the laser radiation can be conveyed into the vein also via a simple optical fiber or a plurality of optical fibers, or else via a catheter without thermal sensors, even though the presence of sensors facilitates control of the treatment.

The mechanism of action contemplates three distinct temporal steps:
step of sclerosis: immediate hardening of the median coat, by direct photothermal effect (shrinkage or hyalinizing sclerosis);
hyperplastic step: fibroblastic-myocellular stimulation, by the photochemical effect of the laser, hyperplasia of the media;
reparative step: remodeling of the venous wall according to the new venous architecture.

These three steps are described in greater detail in what follows. The step of sclerosis has the aim of inducing a temporary hyalinizing sclerosis with structural modifications both to the collagen fibers (shrinkage) and to the extracellular matrix (ECM) of the media, without this involving endothelial damage, i.e., damage to the intima, or else stress to the morphological component of the media and adventitia, i.e., of the outer coats of the tissues forming the treated blood vessel.

These structural modifications intervene immediately during laser treatment. The effect is visible with the naked eye: the treated vessel is coarctated or shrunk, with a significant reduction in its diameter. In the less serious cases, Classes II and III of the CEAP classification, the aesthetic effect is excellent. It is possible to obtain a hyalinizing sclerosis confined just to the median coat by virtue of the optical characteristics of the wavelength of the holmium laser (corresponding to 2100 nm). Since holmium has a high coefficient of tissue absorption, it performs quite a superficial photothermal effect. The reason that there is a photothermal effect confined to the media, without there being any damage to the endothelium and in general to the intima lies in the different mechanisms of dissipation of the heat that the intima and the media have. In fact, the intima is immediately cooled by convection by the blood circulating in the vessel being treated, whereas the media undergoes the phenomenon of thermal accumulation in so far as the heat developed propagates slowly outwards by conduction.

The reason why the morphological component of the media, myocytes and fibroblasts, do not undergo a significant thermal stress is to be sought, rather than in the temperatures involved (45-60° C.), in the extremely short times of exposure of the tissue to the laser radiation, obtained both by pulsing the laser and by moving the fibers in the vein at an appropriate rate, as indicated above.

The photothermal effect induces a structural modification above all to the type III collagen of the media, which undergoes the phenomenon of “shrinkage”. In practice, there is noted the breaking of the hydrogen bonds between the various collagen fibers and their reconstitution in anomalous positions. This structural disorganization leads to a variation of collagenic hydrophilia, which results in a reduced elasticity of the structure itself.

As regards the hyperplastic step, whereas the effect of sclerosis is correlated to the sharp thermal increase of the vessel wall, this second step is dependent upon the interaction of the laser light with the irradiated structures. The light diffuses in all directions and inevitably stimulates the tissue involved. Some authors define it as photochemical effect. In practice, there is noted an increase in the metabolic activity, along with an increase in the synthesis of extracellular matrix, above all in the median coat. In some cases there is evident also an anti-inflammatory effect on the chronic flogistic component, which, however, is counterbalanced, above all in the first hours, by the flogistic effect induced by photothermal stress.

In the days subsequent to the laser treatment, there is observed an increase in the mitotic activity with a reparative evolution typical of the reparative-biostimulant effect of the laser.

The last step, namely the reparative step, is the most important step from the therapeutic standpoint. Its onset starts approximately two weeks after the treatment and involves remodeling of the treated wall by the morphological vasal component: myocytes and fibrocytes. The metabolic turnover envisages the digestion of the extracellular structures, matrix and modified collagen, and their substitution with physiological elements oriented, however, according to lines of force close to the physiological ones. The vessel recovers in time its own elasticity, and the venous system can thus tend towards an albeit partial restitutio ad integrum.

Basically, then, and unlike the destructive techniques so far known, the device and the method according to the invention enable a mini-invasive intervention, which sets itself the aim of conservation of the vessel and of its functional recovery. In fact, the conditions of irradiation chosen cause, on the one hand, the restriction of the median coat without the involvement of the endothelium and of the functional part of the median coat itself, whilst, on the other, they induce an effect of photostimulation of the median coat, which favors its subsequent recovery.

1. A device for the treatment of varicose veins, including a laser source and at least one optical-fiber means for conveying the laser radiation to the vein, wherein the laser source has characteristics of emission such as to cause a hyalinizing sclerosis with structural modifications both to fibers of the collagen (shrinkage) and to the extracellular matrix of the median coat of the vein by the photothermal effect, substantially without thermal stress of the morphological component of the tunica media and of the tunica intima.

2. The device according to claim 1, in which said laser source is a pulsed source and has a wavelength comprised between 800 and 2900 nm, and preferably around 2100 nm.

3. The device according to claim 2, in which said laser source is a holmium laser.

4. The device according to claim 1, including a catheter provided with a plurality of optical fibers terminating in one end of the catheter and arranged and made so as to orient the respective beams in a direction inclined outwards with respect to the axis of the catheter.

5. The device according to claim 2, including a catheter provided with a plurality of optical fibers terminating in one end of the catheter and arranged and made so as to orient the respective beams in a direction inclined outwards with respect to the axis of the catheter.

6. The device according to claim 3, including a catheter provided with a plurality of optical fibers terminating in one end of the catheter and arranged and made so as to orient the respective beams in a direction inclined outwards with respect to the axis of the catheter.

7. The device according to claim 4, wherein each of said optical fibers has a terminal portion inclined outwards with respect to the axis of the catheter.

8. The device according to claim 4, wherein the terminal ends of said optical fibers are arranged according to a circular alignment.

9. The device according to claim 4, wherein the terminal portions of said optical fibers are housed between an outer cylindrical sleeve and an inner tubular element of the catheter, which are coaxial to one another.

10. The device according to claim 4, including a plurality of thermal sensors associated to the end of the catheter.

11. The device according to claim 10, wherein said thermal sensors are arranged on elongated elastic elements, having a movement of extraction and retraction with respect to a terminal housing associated to the end of the catheter.

12. The device according to claim 11, wherein said elongated elastic elements are shaped so as to bend outwards radially when they are extracted from the terminal end of the catheter.

13. The device according to claim 9, wherein said elastic elements are housed in said inner tubular element and can be extracted therefrom.

14. The device according to claim 10, including a control unit of the laser source interfaced to said thermal sensors, for control of the laser source according to the temperature detected by said sensors.

15. The device according to claim 14, wherein said laser source is controlled in such a way as to maintain the temperature of the internal surface of the vessel below 85° C., and preferably below 65° C., and even more preferably between 45° C. and 60° C.

16. The device according to claim 1, wherein said laser source is pulsed at a frequency comprised between 1 and 50 Hz, and preferably between 2 and 25 Hz, and even more preferably between 5 and 20 Hz.

17. The device according to claim 16, wherein said laser source is pulsed at a frequency comprised between 5 and 15 Hz, and preferably between 6 and 10 Hz, and even more preferably between 6 and 8 Hz.

18. The device according to claim 1, wherein said laser source emits at a power comprised between 0.5 and 10 W, and preferably between 1 and 8 W, and even more preferably between 1 and 5 W.

19. The device according to claim 1, wherein each pulse of said laser source has an energy comprised between 50 and 2000 mJ, and preferably between 120 and 900 mJ, and even more preferably between 150 and 700 mJ.

20. An angiological catheter for the treatment of varicose veins, including a plurality of optical fibers terminating in one end of the catheter and arranged and made so as to orient the respective beams in a direction inclined outwards with respect to the axis of the catheter.

21. The catheter according to claim 20, wherein each of said optical fibers has a terminal portion inclined outwards with respect to the axis of the catheter.

22. The catheter according to claim 20, wherein the terminal ends of said optical fibers are arranged according to a circular alignment.

23. The catheter according to claim 21, wherein the terminal ends of said optical fibers are arranged according to a circular alignment

24. The catheter according to claim 20, wherein the terminal portions of said optical fibers are housed between an outer cylindrical sleeve and an inner tubular element, which are coaxial to one another.

25. The catheter according to claim 20, including a plurality of thermal sensors associated to the end of the catheter.

26. The catheter according to claim 24, including a plurality of thermal sensors associated to the end of the catheter.

27. The catheter according to claim 25, wherein said thermal sensors are arranged on elongated elastic elements, having a movement of extraction and retraction with respect to a terminal housing associated to the end of the catheter.

28. The catheter according to claim 27, wherein said elongated elastic elements are shaped so as to bend outwards radially when they are extracted from the terminal end of the catheter.

29. The catheter according to claim 24, wherein said elastic elements are housed in said inner tubular element and can be extracted therefrom.

30. A method for the curative treatment of varicose veins, including application to the wall of a diseased vein of a laser radiation that causes a hyalinizing sclerosis, by direct photothermal effect, to the extracellular matrix substantially limited to the median coat of the vein, without thermal stress of the morphological component of the tunica media and of the tunica intima.

31. A method for the curative intravascular treatment of varicose veins including the following steps:
percutaneous introduction of a wave-guide into the vein to be treated;
irradiation, through said wave-guide, of the wall of said vein with a laser radiation that causes a hyalinizing sclerosis, by direct photothermal effect, with structural modifications both to fibers of the collagen (shrinkage) and to the extracellular matrix substantially limited to the median coat of the vein, without thermal stress of the morphological component of the tunica media and of the tunica intima; and
sliding of said wave-guide during emission of the laser radiation along the stretch of the diseased vein.

32. The method according to claim 31, wherein said sliding of the wave-guide in the vein occurs in a proximal-to-distal direction.

33. The method according to claim 30, wherein said laser radiation has a wavelength comprised between 800 and 2900 nm, and preferably around 2100 nm.

34. The method according to claim 31, wherein said laser radiation has a wavelength comprised between 800 and 2900 nm, and preferably around 2100 nm.

35. The method according to claim 32, wherein said laser radiation has a wavelength comprised between 800 and 2900 nm, and preferably around 2100 nm.

36. The method according to claim 30, wherein said laser radiation is pulsed.

37. The method according to claim 36, wherein said laser radiation is pulsed at a frequency comprised between 1 and 50 Hz, and preferably between 2 and 25 Hz, and even more preferably between 5 and 20 Hz.

38. The method according to claim 37, wherein said laser radiation is pulsed at a frequency comprised between 5 and 15 Hz, and preferably between 6 and 10 Hz, and even more preferably between 6 and 8 Hz.

39. The method according to claim 36, wherein each laser pulse has an energy comprised between 50 and 2000 mJ, and preferably between 120 and 900 mJ, and even more preferably between 150 and 700 mJ.

40. The method according to claim 37, wherein each laser pulse has an energy comprised between 50 and 2000 mJ, and preferably between 120 and 900 mJ, and even more preferably between 150 and 700 mJ.

41. The method according to claim 38, wherein each laser pulse has an energy comprised between 50 and 2000 mJ, and preferably between 120 and 900 mJ, and even more preferably between 150 and 700 mJ.

42. The method according to claim 30, wherein said laser radiation has a power comprised between 0.5 and 10 W, and preferably between 1 and 8 W, and even more preferably between 1 and 5 W.

43. The method according to claim 36, wherein said laser radiation has a power comprised between 0.5 and 10 W, and preferably between 1 and 8 W, and even more preferably between 1 and 5 W.

44. The method according to claim 30, wherein the laser irradiation is controlled so as not to cause damage to the morphological component of the median coat of the treated vein and to the intima.

45. The method according to claim 30, wherein the temperature of the internal surface of the treated vein is kept below 85° C., and preferably below 65° C., and even more preferably is comprised between 45° C. and 60° C.

46. The method according to claim 36, wherein the temperature of the internal surface of the treated vein is kept at a temperature below 85° C., and preferably below 65° C., and even more preferably is comprised between 45° C. and 60° C.

47. The method according to claim 30, wherein the collagen of the median coat of the treated vein is subjected to a coarctation (shrinkage) as a result ofthe breaking of the hydrogen bonds between the collagen fibers caused by the photothermal effect of the laser.

48. The method according to claim 30, including a step of fibroblastic-myocellular photo-stimulation of the median coat of the vein via laser radiation.

49. The method according to claim 30, wherein application of the laser radiation is performed, in the absence of significant thermal stress, prevalently on the morphological component of the median coat.

50. The method according to claim 30, wherein application of the laser radiation is controlled so as to preserve the endothelium.

51. The method according to claim 30, wherein said laser radiation has a wavelength such as to localize the absorption of the radiation prevalently in the median coat of the vein.

52. The method according to claim 30, wherein said laser radiation is generated by a holmium laser.

53. The method according to claim 31, wherein said laser radiation is conveyed within the vein via at least one optical fiber.

54. The method according to claim 31, wherein said laser radiation is conveyed within said vein via a plurality of optical fibers arranged around an axis of a catheter.

55. The method according to claim 30, including the step of monitoring the temperature of the internal surface of the wall of the vein during treatment and of controlling the laser emission according to the temperature detected to maintain said temperature within a pre-determined range.

56. The method according to claim 36, wherein the collagen of the median coat of the treated vein is subjected to a coarctation (shrinkage) as a result of the breaking of the hydrogen bonds between the collagen fibers caused by the photothermal effect of the laser.

57. The method according to claim 36, including a step of fibroblastic-myocellular photo-stimulation of the median coat of the vein via laser radiation.

58. The method according to claim 36, wherein application of the laser radiation is performed, in the absence of significant thermal stress, prevalently on the morphological component of the median coat.

59. The method according to claim 36, wherein application of the laser radiation is controlled so as to preserve the endothelium.

60. The method according to claim 36, wherein said laser radiation has a wavelength such as to localize absorption of the radiation prevalently in the median coat of the vein.

61. The method according to claim 36, wherein said laser radiation is generated by a holmium laser.

62. The method according to claim 36, wherein said laser radiation is conveyed within the vein via at least one optical fiber.

63. The method according to claim 36, wherein said laser radiation is conveyed within said vein via a plurality of optical fibers arranged around an axis of a catheter.

64. The method according to claim 36, including the step of monitoring the temperature of the internal surface of the wall of the vein during treatment and of controlling the laser emission according to the temperature detected to maintain said temperature within a pre-determined range.
